(19) United States
(12) Patent Application Publication
(10) Pub. No.: US 2022/0257108 A1
(43) Pub. Date: Aug. 18, 2022

(54) ENDOSCOPE DEVICES, SYSTEMS, AND METHODS FOR REDUCING INTRA-PELVIC PRESSURE USING SAME

(71) Applicant: The Research Foundation for the State University of New York, Albany, NY (US)

(72) Inventor: Scott Wiener, Camillus, NY (US)

(21) Appl. No.: 17/674,742

(22) Filed: Feb. 17, 2022

Related U.S. Application Data
(60) Provisional application No. 63/150,163, filed on Feb. 17, 2021.

(51) Int. Cl. A61B 1/32; A61B 1/307

(52) U.S. Cl. CPC A61B 1/32; A61B 1/307

(57) ABSTRACT

The present disclosure relates to methods of performing ureteroscopy, a ureteroscope for insertion into a ureter or an access sheath, and systems related thereto. In embodiments, the present disclosure includes an endoscope, for insertion into a ureter or an access sheath, the endoscope including: a shaft including: a first end, a second end positioned axial opposite of the first end, a working channel extending axially through the shaft, between the first end and the second end, and at least one protrusion extending at least one of: from an exterior surface of the shaft, or into the working channel; and a handpiece releasably coupled to the shaft adjacent the first end.

### CROSS-REFERNCE TO RELATED APPLICATIONS

The present disclosure claims priority or the benefit under 35 U.S.C. § 119 of U.S. provisional application No. 63/150,163 filed 17 Feb. 2021, which is herein entirely incorporated by reference.

### FIELD OF THE INVENTION

The present disclosure relates generally to fluid flow during endoscopic procedures, and more particularly to endoscope devices, systems, and methods for improving fluid flow and reducing intra-pelvic pressure during endoscopic procedures.

### BACKGROUND

Ureteroscopy is a method for treatment of nephrolithiasis, including removal of kidney stones, renal calculi and urinary stones, using ureteroscopes. Modern ureteroscopes have reduced in size and become technologically advanced in order to facilitate access of a patient's renal pelvis via a ureter to remove stones. An important medical consideration during ureteroscopy is the development of exceedingly high levels of intra-pelvic pressure (IPP) within the renal pelvis due to an accumulation of irrigation fluid, which places the patient at heightened risk for complications caused by pyelovenous backflow including infection, injury, and pain.

A ureteral access sheath (UAS) placed inside the ureter is one method which may be employed to limit the development of unsafe IPP during ureteroscopy. Experimental evidence suggests that the diameter of the UAS is one critical parameter affecting the fluid outflow rate during ureteroscope insertion, and that increasing the inner diameter of the access sheath is effective at minimizing IPP. However, a major limitation is that selection of the sheath diameter is limited by the diameter of the patient's ureter. Use of a smaller ureteroscope, with or without a UAS present, may be desirable, but technical limitations require a certain minimal cross-sectional area of a ureteroscope to house the internal components and retain functionality.

There is a continuous need for ureteroscopes that overcome these limitations.

### SUMMARY

The present disclosure now provides methods of performing ureteroscopy, a ureteroscope for insertion into a ureter or an access sheath, and systems related thereto that overcome challenges relating to the diameter of the ureter.

A first aspect of the disclosure provides an endoscope, for insertion into a ureter or an access sheath. In embodiments, the endoscope includes: a shaft including: a first end, a second end positioned axial opposite of the first end, a working channel extending axially through the shaft, between the first end and the second end, and at least one protrusion extending at least one of: from an exterior surface of the shaft, or into the working channel; and a handpiece releasably coupled to the shaft adjacent the first end.

A second aspect of the disclosure provides an access sheath for receiving an endoscope. The access sheath including: a conduit including: a first end, a second end positioned opposite of the first end, an opening extended substantially between the first end and the second end, the opening receiving the endoscope; and at least one projection extending radially into the opening of the conduit to increase a flow of fluid at least one of through the opening of the conduit or around the endoscope positioned within the opening of the conduit.

A third aspect of the disclosure provides an endoscope system including: an endoscope including: a shaft including: a first end, a second end positioned axial opposite of the first end, and a working channel extending axially through the shaft, between the first end and the second end; and a handpiece releasably coupled to the shaft of the endoscope adjacent the first end; an access sheath for receiving the endoscope, the access sheath including: a conduit including: a first end, a second end positioned opposite of the first end, and an opening extended substantially between the first end and the second end, the opening receiving the shaft of the endoscope; and at least one of: at least one protrusion included within the shaft of the endoscope, the at least one protrusion extending at least one of: from an exterior surface of the shaft, or into the working channel, or at least one projection extending radially into the opening of the conduit, wherein the at least one protrusion and the at least one projection increase a flow of fluid at least one of through the opening of the conduit or around the endoscope positioned within the opening of the conduit.

A fourth aspect of the disclosure provides a method of performing an endoscopic procedure. The method including: providing an endoscope system including at least one of an endoscope and a ureteral access sheath receiving the endoscope, wherein the endoscope system includes at least one of at least one protrusion included within the shaft of the endoscope, or at least one projection extending radially into the opening of the conduit of the access sheath, and wherein the at least one protrusion and the at least one projection increase a flow of fluid at least one of through the opening of the conduit or around the endoscope positioned within the opening of the conduit of the access sheath.

A fifth aspect of the present disclosure includes an endoscope shaft including: a first end, a second end positioned axial opposite of the first end, at least one working channel extending axially through the shaft, between the first end and the second end, and at least one protrusion extending at least one of: from an exterior surface of the shaft, or into the working channel. In embodiments, the endoscope shaft is fixedly attached to a handpiece releasably coupled to the shaft adjacent the first end. In embodiments, one or more protrusion(s) extend(s) along a length of the exterior surface of the shaft from the first end to the second end. In embodiments, one or more protrusions extend along one or more segments of the exterior surface of the shaft from the first end to the second end. In embodiments, the one or more segments extend along the same length of the exterior surface, such as in a line. In embodiments, the one or more segments extend along different lengths of the exterior surface and are separated by a variable or fixed length.

The illustrative aspects of the present disclosure are designed to solve the problems herein described and/or other problems not discussed.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

FIG. 1 shows a side view of an endoscope system including an endoscope and an access sheath according to embodiments of this disclosure.

FIG. 2 shows a front cross-sectional view of the endoscope taken along the line of 2-2 of FIG. 1, according to embodiments of the disclosure.

FIG. 3 shows a side view of an endoscope system including an endoscope and an access sheath according to additional embodiments of this disclosure.

FIG. 4 shows a front cross-sectional view of the endoscope taken along the line of 4-4 of FIG. 3, according to embodiments of the disclosure.

FIG. 5 shows a front cross-sectional view of an endoscope, according to additional embodiments of the disclosure.

FIG. 6 shows a front cross-sectional view of an endoscope, according to further embodiments of the disclosure.

FIG. 7 shows a side view of an endoscope for an endoscope system according to further embodiments of this disclosure.

FIG. 8 shows a front cross-sectional view of an endoscope and an access sheath, according to embodiments of the disclosure.

FIGS. 9 and 10 show front cross-sectional views of an endoscope and an access sheath, according to additional embodiments of the disclosure.

FIG. 11 shows a side view of an endoscope system including an endoscope and an access sheath according to embodiments of this disclosure.

FIG. 12 show front cross-sectional view of the endoscope and the access sheath taken along the line of 12-12 of FIG. 11, according to additional embodiments of the disclosure.

FIG. 13 shows a side view of an endoscope for an endoscope system, according to embodiments of the invention.

FIG. 14 shows a side view of an endoscope system including an endoscope and an access sheath, according to embodiments of the invention.

FIG. 15 show a flowchart illustrating a process for performing an endoscopic procedure to improve fluid flow and reduce intra-pelvic pressure, according to embodiments of the disclosure.

FIG. 16 shows representation diameters, resistance to fluid flow, and cross-sectional area of four different access sheath shapes (i-iv). FIG. 16 also depicts two graphs (1-2) showing intra-pelvic pressure (IPP) and fluid flow rate (outflow) for various measurements of irrigation pressure for those four shapes, according to embodiments of the disclosure.

FIG. 17 shows a side view of an endoscope for an endoscope system, according to embodiments of the disclosure.

FIG. 18 shows a front cross-sectional view of an endoscope, according to further embodiments of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION

The present disclosure now provides methods of performing ureteroscopy, a ureteroscope for insertion into a ureter or an access sheath, an endoscope shaft, and systems related thereto that overcome challenges relating to the diameter of the ureter.

In embodiments the present disclosure includes an endoscope, for insertion into a ureter or an access sheath. In embodiments, the endoscope includes: a shaft including: a first end, a second end positioned axial opposite of the first end, a working channel extending axially through the shaft, between the first end and the second end, and at least one protrusion extending at least one of: from an exterior surface of the shaft, or into the working channel. In embodiments, the endoscope includes a handpiece releasably coupled to the shaft adjacent the first end.

In some embodiments the present disclosure includes an endoscope shaft, for insertion into a ureter or an access sheath. In embodiments, the endoscope shaft includes: a first end, a second end positioned axial opposite of the first end, a working channel extending axially through the shaft, between the first end and the second end, and at least one protrusion extending at least one of: from an exterior surface of the shaft, or into the working channel.

Advantages of the endoscopes of the present disclosure is that predetermined shapes of the shaft and/or access sheath are provided for improving fluid flow and reducing intra-pelvic pressure during endoscopic procedures.

### Definitions

As an initial matter, in order to clearly describe the current disclosure it will become necessary to select certain terminology when referring to and describing relevant components within the disclosure. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

As used herein, the terms “axial” and/or “axially” refer to the relative position/direction of objects along axis (A), which is substantially parallel with the long axis of the circular shafts, conduits, tubes, channels, etc. discussed herein.

As used herein, the terms “radial” and/or “radially” refer to the relative position/direction of objects along axis (R), which is substantially perpendicular with axis (A) and intersects axis (A) at only one location.

As used herein the terms “circumferential” and/or “circumferentially” refer to the relative position/direction (C) of objects or features along a circumference which surrounds axis (A) but does not intersect the axis (A) at any location.

### DETAILED DISCLOSURE OF CERTAIN EMBODIMENTS

As discussed herein, the disclosure relates generally to fluid flow during endoscopic procedures, and more particularly to endoscope devices, systems, and methods for improving fluid flow and reducing intra-pelvic pressure during endoscopic procedures.

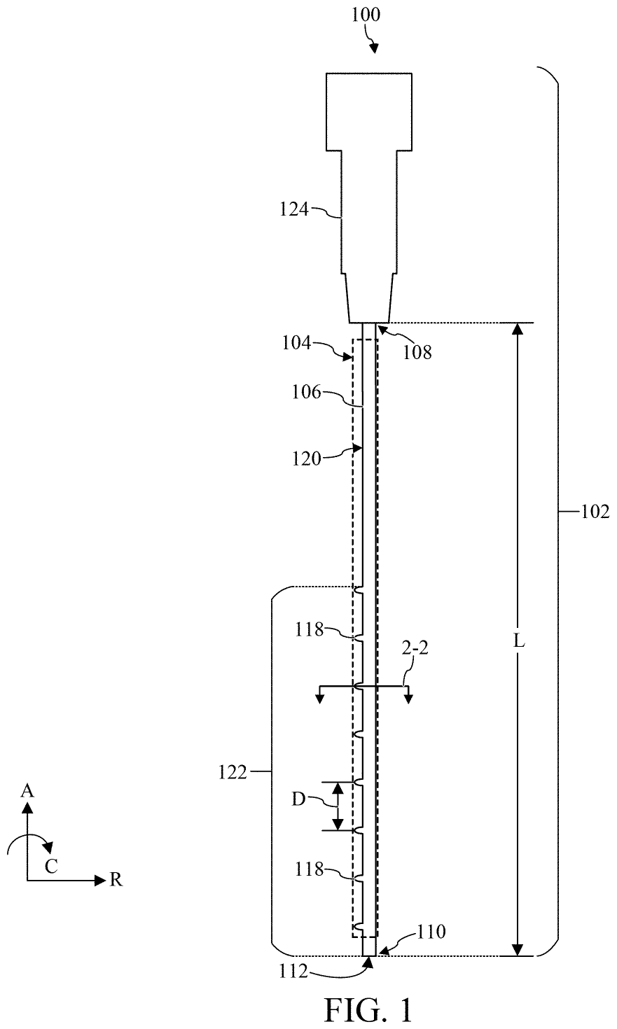

These and other embodiments are discussed below with reference to FIGS. 1-18. However, those skilled in the art will readily appreciate that the detailed description given herein with respect to these Figures is for explanatory purposes only and should not be construed as limiting.

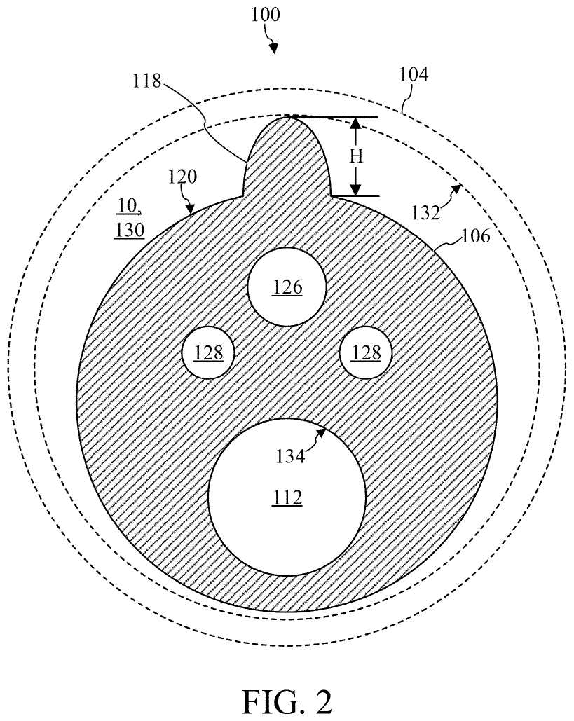

FIG. 1 shows a side view of an endoscope system 100. Endoscope system 100 may be used to perform endoscopic procedures and/or processes on a patient or user such as a process that contacts the endoscope system with a patient in need thereof. In a non-limiting embodiment endoscope system 100 may include an endoscope device 102 and an access sheath 104 for receiving at least a portion of endoscope device 102. As shown in FIG. 1, access sheath 104 as shown in phantom as optional. As discussed herein, access sheath 104 may or may not be used when performing an endoscopic procedure on a patient using endoscope 102. That is, in some endoscopic procedures endoscope 102 may be directly inserted into a lumen of a patient in need thereof, while in another endoscopic procedure embodiment access sheath 104 may be inserted into a patient's lumen 10 (see, FIG. 2) and endoscope 102 may subsequently be inserted into access sheath 104. As such, some of the figures herein may depict a patient's lumen (see, FIG. 2) or alternatively an opening of access sheath 104—each receiving endoscope directly.

In embodiments, endoscope 102 shown in FIG. 1 may include a shaft 106 having a first end 108 and a second end 110 positioned axially opposite first end 108. As discussed herein, shaft 106 may also include a working channel 112 extending axially through shaft 106, between first end 108 and second end 110. Shaft 106 of endoscope 102 may include a predetermined length (L). Predetermined length (L) of shaft 106 may be based on, at least in part, the endoscopic procedure performed on the patient, and/or physical characteristics of the patient (e.g., sex of patient, age of patient, patient's lumen for receiving endoscopic device 102, and so on). In a non-limiting example, predetermined length (L) of shaft 106 of endoscope 102 may be between approximately 50 centimeters (cm) and approximately 100 centimeters (cm), or between approximately 60 cm and 70 cm. Shaft 106 of endoscope 102 may be formed from any suitable material that may be flexible but maintain its shape and/or it's rigidity during the endoscopic procedure. For example, shaft 106 may be formed from a polymer or polymer-based material. In embodiments, the shaft 106 may be formed from a latex-free polymer material.

In embodiments, shaft 106 of endoscope 102 may also include at least one protrusion 118. In a non-limiting example shown in FIG. 1, at least one protrusion 118 may extend from an exterior surface 120 of shaft 106. As shown in the non-limiting example, shaft 106 of endoscope 102 may include a plurality of protrusions extending from exterior surface 120 of shaft 106. For example, in embodiments, 1-100, 1-50, or 1-30, 1-20, 1-10 protrusions may extend from exterior surface 120 of shaft 106. In embodiments, the plurality of protrusions may be positioned in a line atop the exterior surface 120 of shaft 106 from or between the first end 108 and the second end 110. A non-limiting example is shown in FIG. 1, wherein eight protrusions are shown in a line directly atop or fixedly attached to the exterior surface 120 of shaft 106, the line formed between the first end 108 and the second end 110. In embodiments, each of the plurality of protrusions 118 may extend radially from exterior surface 120 of shaft 106. Additionally, and as shown in FIG. 1, the plurality of protrusions 118 may be axially aligned with one another along shaft 106. In the non-limiting example shown in FIG. 1, each of the plurality of protrusions 118 may be integrally formed with and/or within shaft 106, such that shaft 106 and protrusions 118 include a single, unitary body or component. In another non-limiting example, each of the plurality of protrusions 118 may be formed as a distinct component from shaft 106, and may be coupled, connected, and/or fixed to exterior surface 120 of shaft 106. In the example where protrusions 118 are formed integral with shaft 106, each protrusion 118 may be formed from the same material as shaft 106. In the non-limiting example where protrusions 118 are formed as distinct components from shaft 106, each protrusion 118 may be formed from any suitable material that may be substantially rigid, and may or may not be the same material forming shaft 106. For example, protrusions 118 may be formed from polymers, polymer-based material, metals, metal-alloys, ceramics, or any other material having similar characteristics. In embodiments, latex-free polymers are suitable for use herein.

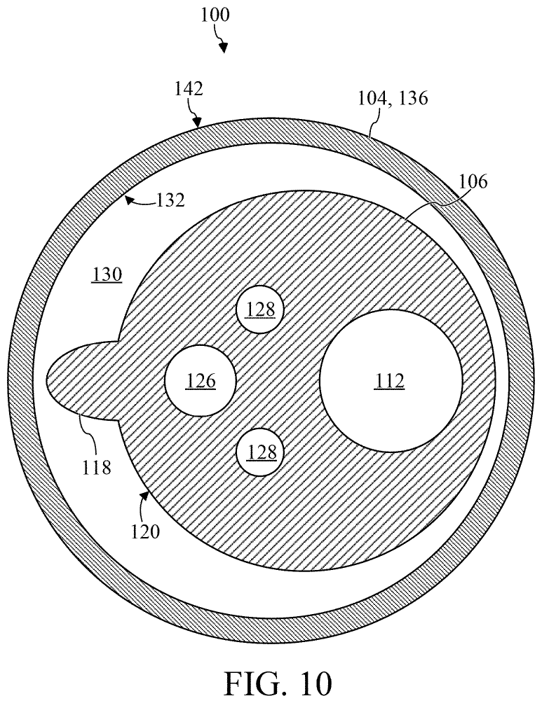

As shown in FIG. 1, and discussed herein, protrusions 118 may be substantially curved in shape or configuration. More specifically, at least a portion of a surface for each of the plurality of protrusions 118 may include a curvature or non-linear shape. The curvature and/or non-linear shape of protrusions 118 may aid in the insertion and/or the movement of endoscope 102, and more specifically shaft 106, within access sheath 104 or a patient's lumen 10 (see, FIG. 10). In the non-limiting example shown in FIG. 1, each protrusion of the plurality of protrusions 118 are axially spaced a predetermined distance (D) from an adjacent protrusion 118. In embodiments, each protrusion 118 may be axially spaced an equal distance (D) from adjacent protrusions 118 extending axially over exterior surface 120 of shaft 106. The predetermined distance (D) may be based on at least in part the predetermined length (L) of shaft 106 of endoscope 102. In a non-limiting example, each protrusion of the plurality of protrusions 118 may be axially spaced between approximately one (1) centimeter (cm) and approximately 10 centimeters (cm) apart from adjacent protrusions 118.

Additionally as shown in FIG. 1, protrusions 118 may be formed substantially adjacent second end 110 of shaft 106. More specifically, at least one protrusion 118 of the plurality of protrusions 118 may be formed on or directly adjacent second end 110, and the remainder of the plurality of protrusions 118 may be formed on and/or may extend from shaft 106 substantially adjacent second end 110. As shown in FIG. 1, the plurality of protrusions 118 may be formed in a predetermined portion 122 of shaft 106. Portion 122 of shaft 106 including the plurality of protrusions 118 may be formed adjacent second end 110, and/or may span over a percentage of the length (L) of shaft 106 adjacent second end 110 and proximate first end 108. The size or length of portion 122 of shaft 106 including protrusions 118 may be dependent, at least in part, on the predetermined length (L) of shaft 106, the endoscopic procedure being performed using endoscope 102, and/or patient characteristics.

In the non-limiting example shown in FIG. 1, endoscope 102 of endoscope system 100 may also include a handpiece 124. Handpiece 124 may be releasably coupled to shaft 106. More specifically, handpiece 124 may be releasably coupled to first end 108 of shaft 106 of endoscope 102. As discussed herein, handpiece 124 may provide access to and/or may allow a user to control features, devices, and/or apparatuses that may extend through shaft 106 and used in performing the endoscopic procedure (e.g., camera, lights, removal baskets, etc.).

Referring now to FIG. 2, a front cross-sectional view of shaft 106 of endoscope 102 for endoscope system 100 is shown. The cross-section of you of shaft 106 may be taken along line 2-2 in FIG. 1. It is understood that similarly numbered and/or named components may function in a substantially similar fashion. Redundant explanation of these components has been omitted for clarity.

As shown in FIG. 2, shaft 106 of endoscope 102 may include working channel 112 extending therethrough. Working channel 112 may extend through shaft 106 between first end 108 and second end 110. Additionally as shown, working channel 112 may be formed within shaft 106 offset or off-center from exterior surface 120 of shaft 106. That is, working channel 112 may be non-concentric with the substantially circular geometry of exterior surface 120 of shaft 106. Working channel 112 may provide a channel to receive additional components, features, and/or apparatus is that may aid or be used in performing the endoscopic procedure using endoscope 102.

Additionally in the non-limiting example, endoscope 102 may include additional channels or conduits 126, 128 extending wholly, or at least partially through shaft 106. Channel 126 may receive an endoscopic camera that may be utilized in the endoscopic procedure performed on the patient using endoscope system 100. Channel 128 may receive lights or any suitable illumination device to provide light for the camera positioned within channel 126 during the endoscopic procedure using endoscope 102. Although two channels 128 are shown for illumination devices, and one channel 126 is shown for a camera, it is understood that shaft 106 of endoscope 102 may include more or less channels 126, 128.

Still referring to FIG. 2, and as discussed herein, shaft 106 of endoscope 102 may be received by, inserted into, and/or positioned within an opening 130 of access sheath 104 of endoscope system 100, or alternatively may be positioned directly within a lumen 10 of a patient. Protrusions 118 of shaft 106 may be positioned directly adjacent or may contact an inner surface 132 of access sheath 104 that defines opening 130. Additionally as shown, and as a result of the formation of protrusions 118 an exterior surface 120 of shaft 106, shaft 106, and more specifically working channel 112, may be substantially offset from the center of opening 130 of access sheath 104 (or lumen 10 where applicable). Furthermore, shaft 106/working channel 112 may be non-concentric with opening 130 of access sheath 104 (or lumen 10) as a result of forming protrusions 118 an exterior surface 120 of shaft 106. As discussed herein, the position (offset, non-concentricity) of working channel 112/shaft 106 within opening 130/lumen 10 may improve the flow of fluids through working channel 112 of shaft 106, through opening 130 of access sheath 104 (or lumen 10), and/or around shaft 106 of endoscope 102 during the endoscopic procedures performed using endoscope system 100. Additionally, or alternatively, the position of working channel 112/shaft 106 within opening 130/lumen 10 may reduce the intra-pelvic pressure experienced within the renal pelvis during the endoscopic procedures.

In embodiments, the plurality of protrusions extending radially from exterior surface 120 of shaft 106 may include a predetermined height (H). More specifically, each of the plurality of protrusions 118 may extend radially from exterior surface 120 at a predetermined height (H). The predetermined height (H) for protrusions 118 may be dependent upon, at least in part, the size/diameter of shaft 106, the size/diameter of opening 130 of access sheath 104 or lumen 10, the endoscopic procedure performed on the patient, and/or patient characteristics. In embodiments, the predetermined height may be between approximately 0.1 millimeters (mm) and approximately 3.0 millimeters (mm). In embodiments, the predetermined height may be between approximately 0.1 millimeters (mm) and approximately 2.0 millimeters (mm). In embodiments, the predetermined height may be between approximately 0.1 millimeters (mm) and approximately 1.5 millimeters (mm). In the non-limiting example where protrusions 118 extend radially from exterior surface 120 of shaft 106 the predetermined height for each of the plurality of protrusions 118 may be between approximately 0.5 millimeters (mm) and approximately 1.5 millimeters (mm).

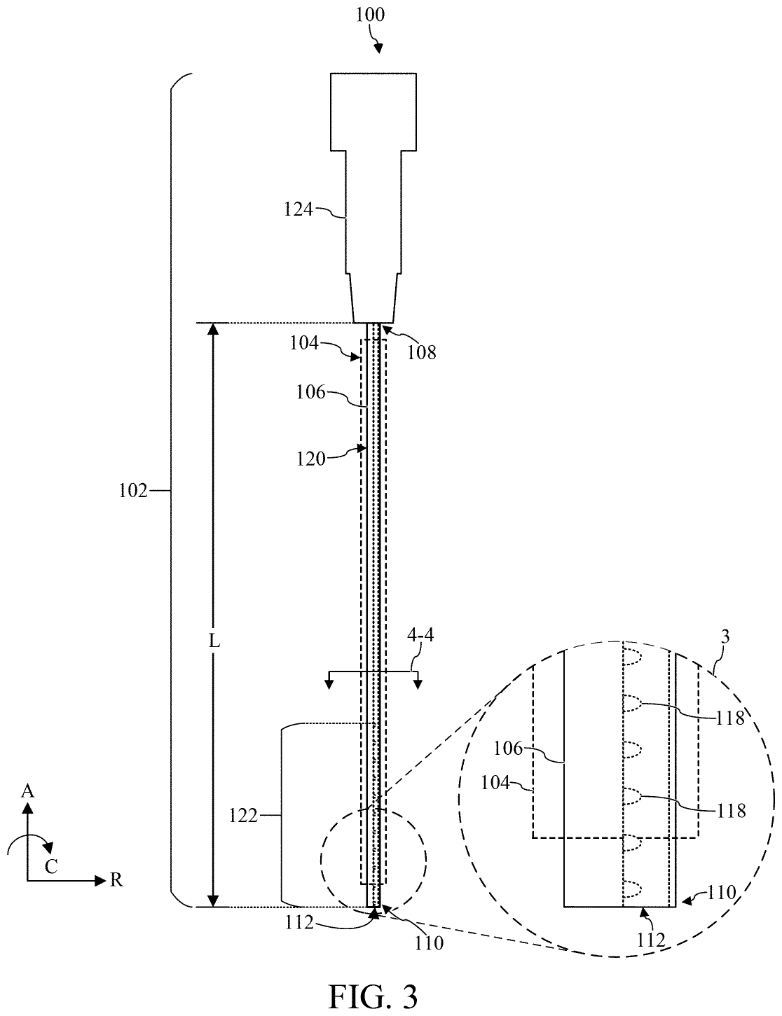

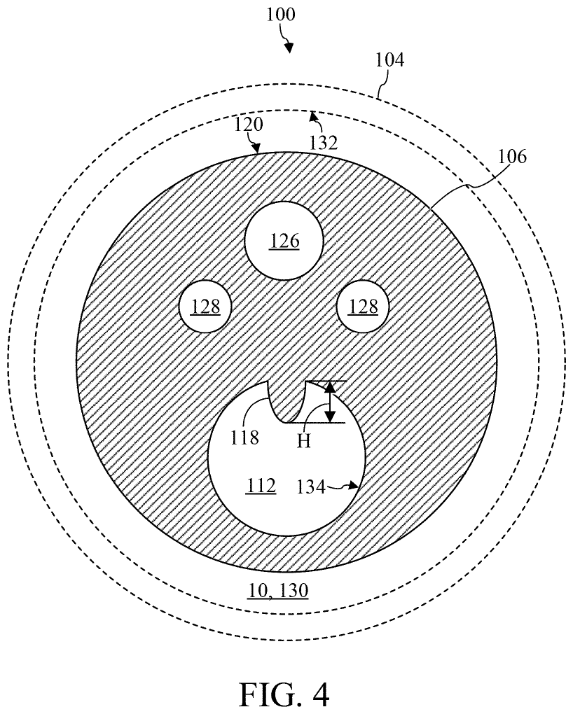

FIGS. 3 and 4 show various views of another non-limiting example of endoscope system 100. More specifically, FIG. 3 shows a side view of endoscope system 100 including an enlarged insert 3, while FIG. 4 shows a front cross-sectional view of shaft 106 of endoscope 102 for endoscope system 100 taken along line 4-4 in FIG. 3. It is understood that similarly numbered and/or named components may function in a substantially similar fashion. Redundant explanation of these components has been omitted for clarity.

With comparison to endoscope 102 in FIG. 1, endoscope 102 shown in FIG. 3, in embodiments, may not include protrusions 118 extending from exterior surface 120 of shaft 106. That is, protrusions 118 included in shaft 106 of endoscope 102 may be positioned, formed, and/or included in a distinct portion of endoscope 102. Turning to FIG. 4, with continued reference to FIG. 3 and enlarged insert 3, protrusions 118 may extend into working channel 112. More specifically, a plurality of protrusions 118 may extend radially inward and/or radially into working channel 112 extending through shaft 106 of endoscope 102. Each protrusion 118 may extend radially into working channel 112 from an interior surface 134 forming and/or defining working channel 112. Similar to endoscope 102 shown and discussed herein with respect to FIG. 2, protrusions 118 extending radially into working channel 112 may include a predetermined height (H). In the non-limiting example shown in FIG. 4, the predetermined height (H) may be between approximately 0.1 millimeters (mm) and approximately 0.5 millimeters (mm). The inclusion of protrusion 118 within working channel 112 may result in working channel 112 being non-circular in shape and/or configuration. Additionally, the inclusion of protrusions 118 in working channel 112 may similarly improve/increase fluid flow through/around endoscope 102, and/or may reduce the intra-pelvic pressure experienced within the renal pelvis during the endoscopic procedures, as discussed herein.

As shown in the non-limiting example of FIGS. 3 and 4, and similar to the example shown and discussed herein with respect to FIGS. 1 and 2, the plurality of protrusions 118 may be axially aligned within working channel 112. Additionally, each of the plurality of protrusions extending into working channel 112 may be actually spaced a predetermined distance (D) from one another, where the predetermined distance (D) is dependent, at least in part, on the length (L) of shaft 106 for endoscope 102. In the non-limiting example, the predetermined distance (D) axially separating adjacent protrusions 118 may be between approximately one (1) centimeter (cm) and approximately 10 centimeters. Furthermore, the plurality of protrusions 118 may be formed in a predetermined portion 122 of working channel 112 extending through shaft 106. Portion 122 of working channel 112 including the plurality of protrusions 118 may be formed adjacent second end 110, and/or may span over at least a percentage of the length (L) of shaft 106 adjacent second end 110 and proximate first end 108.

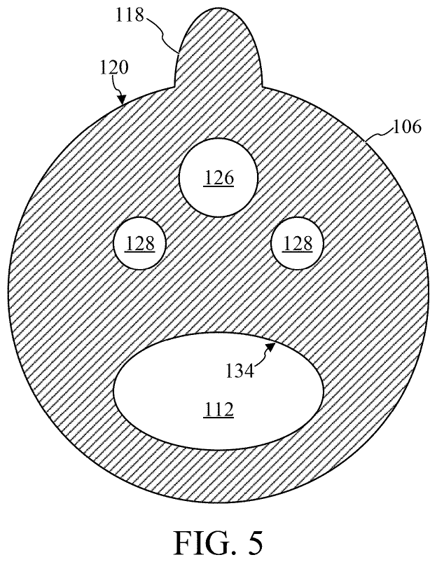

FIG. 5 shows another non-limiting example of endoscope 102. Specifically, FIG. 5 shows a front cross-sectional view of endoscope 102 of endoscope system 100. In the non-limiting example, working channel 112 extending through shaft 106 of endoscope 102 may include a non-circular configuration. For example, working channel 112 shown in FIG. 5 may be formed or configured as an ellipse. In the example, protrusion 118 may extend from exterior surface 120 of shaft 106, and no protrusion may be formed or extend into working channel 112. In another non-limiting example, protrusion 118 may extend into non-circular working channel 112 and may or may not extend from exterior surface 120 of shaft 106.

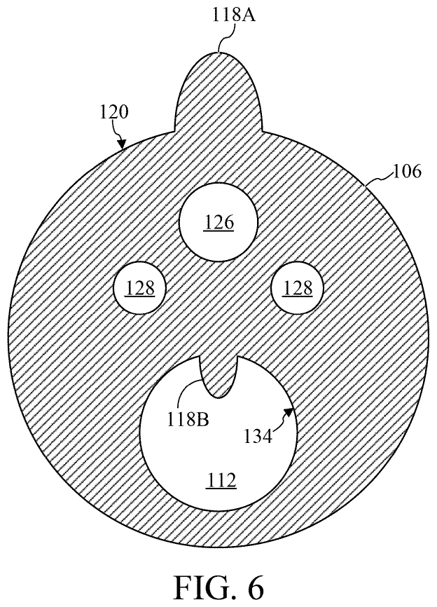

FIG. 6 shows an additional non-limiting example of endoscope 102. In the non-limiting example, shaft 106 may include two sets of protrusions 118A, 118B. More specifically, shaft 106 may include a first set of protrusions 118A extending radially from exterior surface 120 of shaft 106, and a second set of protrusions 118B extending radially into working channel 112 of shaft 106.

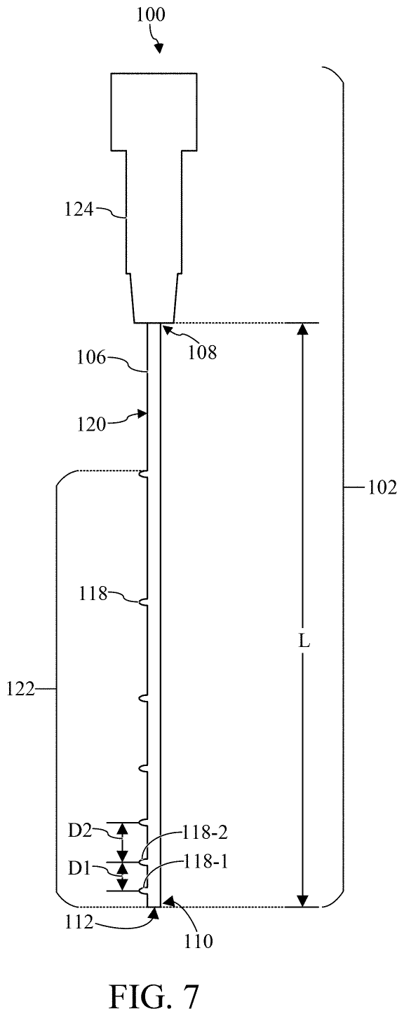

FIG. 7 shows a side view of endoscope system 100 including endoscope 102 and handpiece 124. Endoscope 102 shown in FIG. 7 may be substantially similar and/or may include substantially similar features as though shown and discussed herein with respect to FIGS. 1 and/or 2. Distinct from the embodiment shown in FIG. 1, protrusions 118 extending radially from exterior surface 120 of shaft 106 may not be evenly spaced apart by the predetermined distance (D). Rather the space or distance between each of the plurality of protrusions 118 may vary. For example, a first protrusion118-1 and a second protrusion 118-2 may be axially spaced a first predetermined distance (D1), while the second protrusion 118-2 and a third protrusion 118-3 are axially spaced a second predetermined distance (D2). As shown in FIG. 7, the second predetermined distance (D2) may be distinct (e.g., greater) then the first predetermined distance (D1). Although shown as increasing in axial spacing between protrusions 118 from second end 110 to first end 108, it is understood that the distance axially separating each protrusion 118 of endoscope 102 may vary or be distinct in any suitable manner.

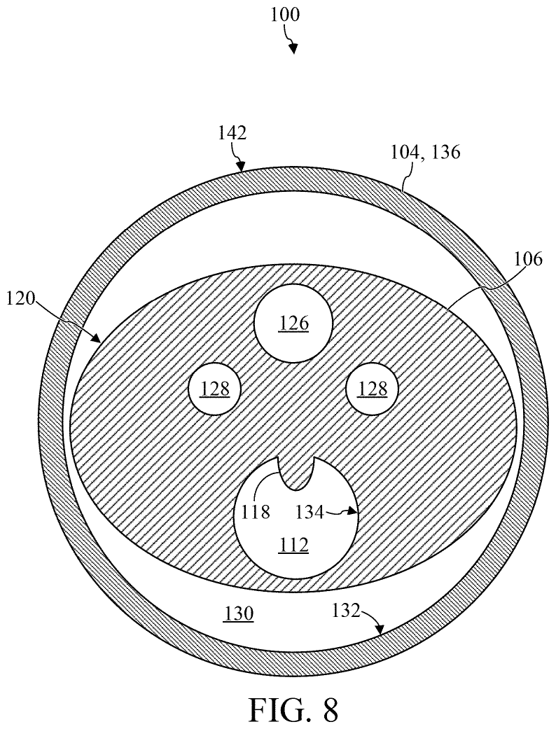

FIGS. 8-10 show additional non-limiting examples of endoscope system 100 where endoscope may include at least one feature formed therein to increase fluid flow through/around endoscope 102, and/or reduce the intra-pelvic pressure experienced within the renal pelvis during the endoscopic procedures, as discussed herein. It is understood that similarly numbered and/or named components may function in a substantially similar fashion. Redundant explanation of these components has been omitted for clarity.

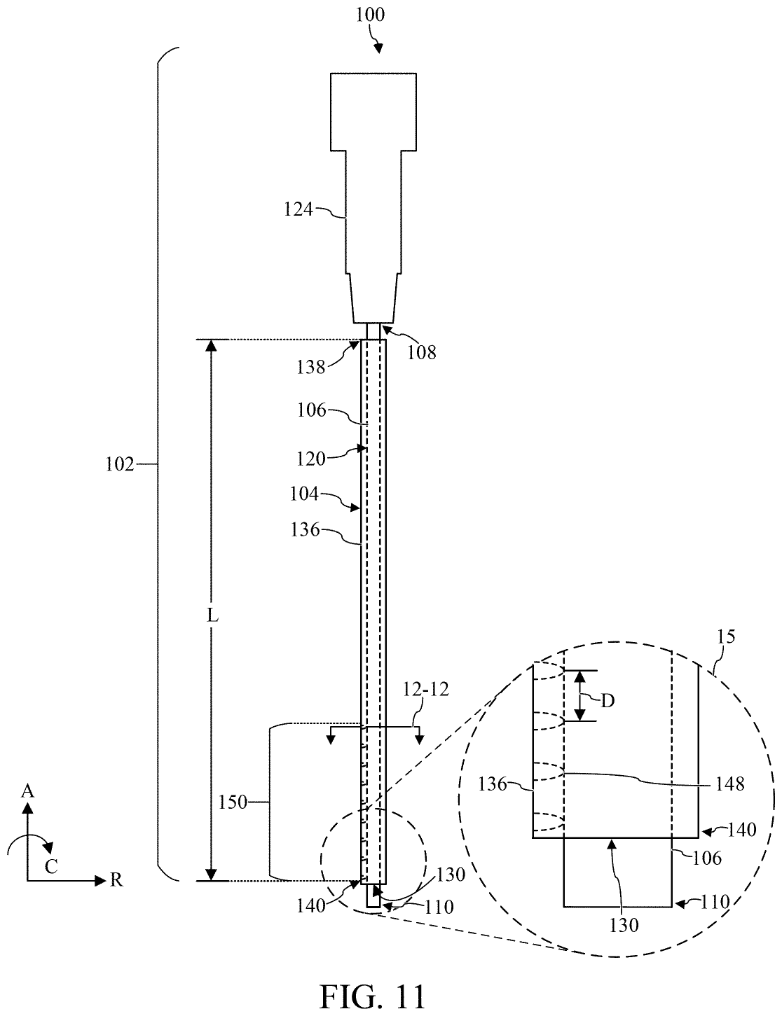

Turning to FIG. 8, access sheath 104 of endoscope system 100 may include or be formed as a conduit 136. Conduit 136 may include a first end 138 and a second end 140 positioned opposite first end 138 (see, FIG. 11). Additionally, and as discussed herein, conduit 136 of access sheath 104 may include opening 130 extending substantially between first end 138 and second end 140. Opening 130 of conduit 136 may receive at least a portion of shaft 106 of endoscope 102. Conduit 136 of access sheath 104 may be formed from any suitable material that may maintain its shape and/or it's rigidity during the endoscopic procedure as to not pinch or constrict movement of endoscope 102 received therein. For example, conduit 136 may be formed from a polymer, polymer-based material, a metal, or a metal-alloy material.

In the non-limiting example shown in FIG. 8 shaft 106 for endoscope 102 may include a non-circular cross-sectional configuration. That is, and as shown in FIG. 8, exterior surface 120 of shaft 106 may include a non-circular, and more specifically an elliptical, cross-sectional configuration or shape. In the non-limiting example, inner surface 132 and outer surface 142 of opening 130 for conduit 136 forming access sheath 104 may be substantially circular.

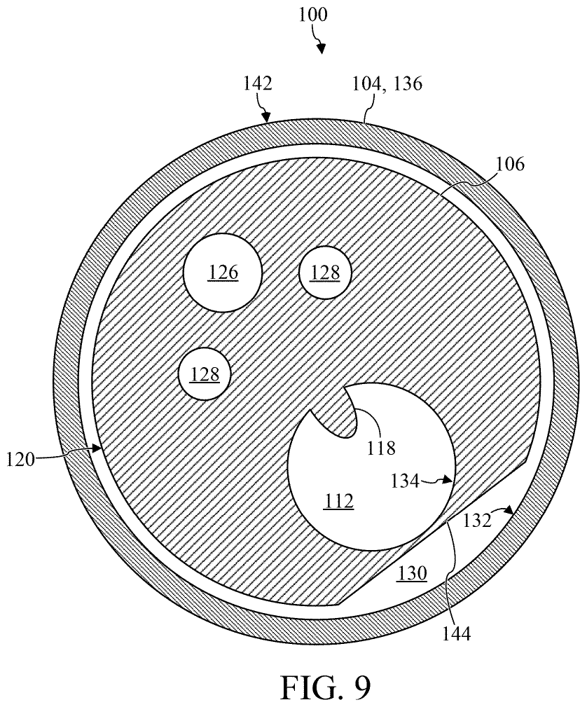

FIG. 9 shows another non-limiting example where exterior surface 120 of shaft 106 forming endoscope 102 may include a non-circular cross-sectional configuration. In the non-limiting example, the non-circular cross-sectional configuration for shaft 106 may include a segmented circular configuration for exterior surface 120 of shaft 106. That is, and as shown in FIG. 9, exterior surface 120 of shaft 106 for endoscope 102 may include a chord section 144 that linearly extends between circular portions of exterior surface 120. The non-circular (e.g., elliptical, segmented circular) cross-sectional configuration of exterior surface 120 of shaft 106 may increase fluid flow through/around endoscope 102, and/or reduce the intra-pelvic pressure experienced within the renal pelvis during the endoscopic procedures, as discussed herein.

FIG. 10 show additional non-limiting examples of endoscope 102 and access sheath 104 forming endoscope system 100. In the non-limiting examples shown in FIG. 10, shaft 106 of endoscope 102 may be substantially rotated such that protrusion(s) 118 extending radially from exterior surface 120 is positioned adjacent to and/or contacts a side portion of conduit 136 forming access sheath 104. That is, and distinct from the example shown in FIG. 2 where shaft 106 is positioned such that protrusion(s) 118 extending substantially vertically, shaft 106 may be positioned such that protrusion(s) 118 extend substantially horizontally to contact a side portion of conduit 136 of access sheath 104. The position of protrusion(s) 118 shown in FIG. 10 may offset working channel 112 from the center of opening 130 of conduit 136/access sheath 104. That is, working channel 112 may be non-concentric with the substantially circular geometry of opening 130/inner surface 132 of conduit 136.

FIG. 11 shows another non-limiting example of endoscope system 100 including endoscope 102 and access sheath 104. As shown in FIG. 11, and discussed similarly herein with respect to FIG. 8, access sheath 104 may include and/or be formed as conduit 136 including first end 138 and second end 140 position to axially opposite first end 138. Similar to non-limiting examples discussed herein, access sheath 104 may include at least one feature within opening 130 of conduit 136 to increase a fluid flow and/or reduce the pressure during an endoscopic procedure. In the non-limiting example shown in FIG. 11, at least one feature may include at least one projection 148 extending into opening 130 of conduit 136. As shown, access sheath 104/conduit 136 may include a plurality of projections 148. Projections 148 included in/on access sheath 104 may be substantially similar to protrusions 118 formed within shaft 106 of endoscope 102. For example, at least a portion of a surface of projections 148 may include a curvature and/or a curved portion. Additionally, each projection 148 of the plurality of projections included in conduit 136 may be axially aligned and may be axially spaced a predetermined distance (D) from adjacent projections 148. A similarly discussed herein, the predetermined distance may be based on a length of conduit 136 and/or a length of shaft 106 of endoscope 102. The predetermined distance (D) for the axial spacing of adjacent projections 148 may be between approximately one (1) centimeter (cm) and approximately 10 centimeters (cm). Furthermore, each of the plurality of projections 148 may be formed in a predetermined portion 150 of access sheath 104/conduit 136 that may be substantially adjacent second end 140 of conduit 136.

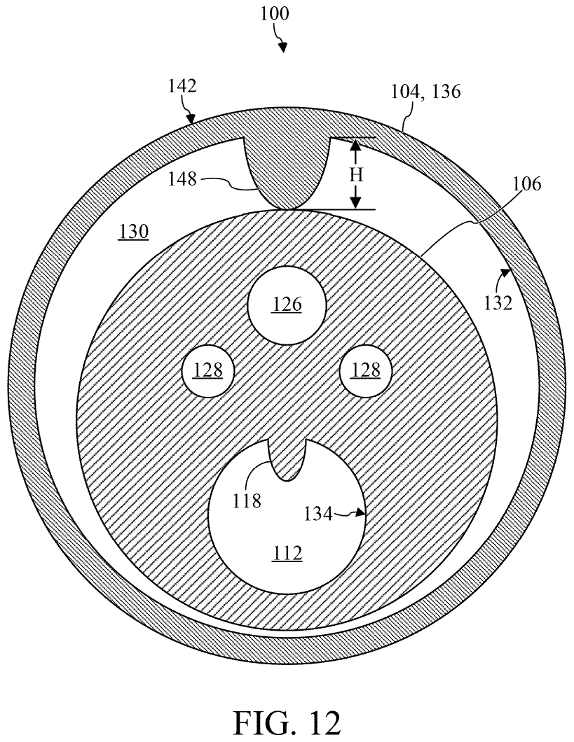

Turning to FIG. 12 with continued reference to FIG. 11, each projection 148 may extend radially into opening 130 of conduit 136. More specifically, projections 148 may extend radially from inner surface 132 of opening 130 for conduit 136 and may be positioned adjacent to or may contact exterior surface 120 of shaft 106 for endoscope 102. A similarly discussed herein with respect to protrusions 118 of endoscope 102, projections 148 included in access sheath 104 may be formed integral with conduit 136, or alternatively may be formed as a separate or distinct component that may be coupled and/or affixed to inner surface 132 of opening 130 for conduit 136.

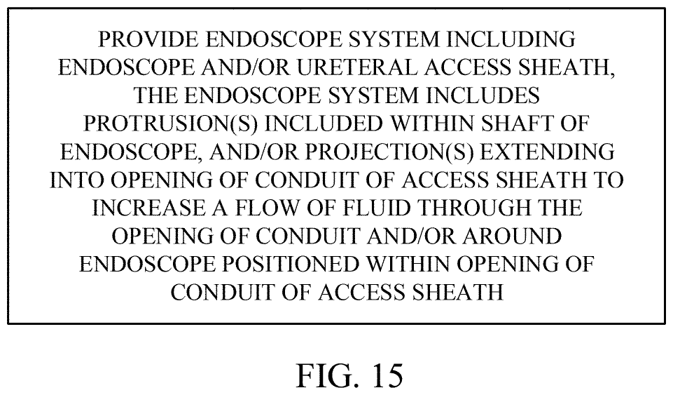

Additionally, the plurality of projections 148 extending radially from inner surface 132 of conduit 136 may include a predetermined height (H). More specifically, each of the plurality of projections 148 may extend radially from inner surface 132 at a predetermined height (H). The predetermined height (H) for projections 148 may be dependent upon, at least in part, the size/diameter of opening 130, the size/diameter of shaft 106 for endoscope 102, the endoscopic procedure performed on the patient, and/or patient characteristics. The predetermined height may be between approximately 0.1 millimeters (mm) and approximately 1.5 millimeters (mm). In the non-limiting example shown in FIG. 15 the predetermined height for each of the plurality of projections 148 may be between approximately 0.5 millimeters and approximately 1.5 millimeters. The plurality of projections 148 formed in access sheath 104 may displace endoscope 102 from a central position within opening 130 of conduit 136. This in turn may increase fluid flow and/or decrease the pressure during the endoscopic procedure performed using endoscope system shown in FIGS. 11 and 12.

Although shown herein as including protrusions 118, it is understood that endoscope 102, and more specifically shaft 106, may or may not require protrusions 118 where conduit 136 of access sheath 104 includes projection 148

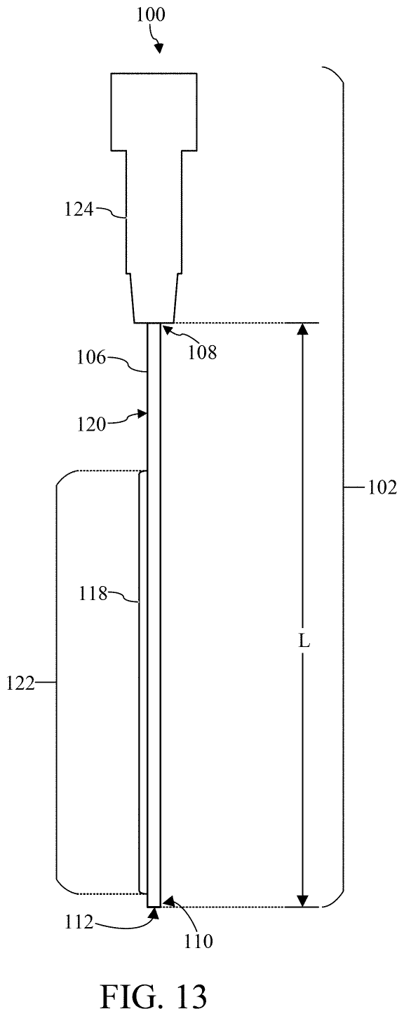

FIG. 13 shows another non-limiting example of endoscope system 100. More specifically, FIG. 13 shows another non-limiting example of endoscope 102 of endoscope system 100. As discussed in some examples herein, endoscope 102 may include at least one protrusion 118 extending radially from exterior surface 120 of shaft 106. As shown, shaft 106 may include a single protrusion 118 that extend over a portion 122 of shaft 106. That is, endoscope 102 may include a single protrusion 118 that extends radially from exterior surface 120 of shaft 106, as well as extends axially over portion 122 of length (L) of shaft 106. In the non-limiting example, single protrusion 118 may be formed substantially adjacent second end 110 of shaft 106 and may extend axially toward and/or proximate first end 108. As similarly discussed herein, the size, length, and/or dimension of portion 122 in which protrusion 118 extends over shaft 106 may be dependent, at least in part, on the length (L) of shaft, the endoscopic procedure to be performed, and/or patient characteristics.

As shown in the non-limiting example, single protrusion 118 may have a substantially uniform shape and/or configuration. As discussed herein, at least a portion of protrusion 118 may include a curved and/or non-linear surface to improve insertion of endoscope 102 into access sheath 104 and/or patient's lumen 10. Additionally, and as similarly discussed herein, single, uniform protrusion 118 may include and/or be formed with a predetermined (H) between approximately 0.1 millimeters (mm) and 1.5 mm. In other non-limiting examples (not shown), the height (H) of single protrusion 118 may vary over the axial length of protrusion 118. For example, protrusion 118 may include a central portion that includes a first height (H1), and two opposing end portions formed on opposing sides of central portion that include a second height (H2). The second height (H2) may be distinct (e.g., less than, greater than) from the first height (H1). The difference in high in each portion may be achieved via a stepped configuration in protrusion 118 or alternatively a gradual incline/decline between end portions and central portion. In another example (not shown), the height (H) of single protrusion 118 may vary multiple times over the axial length (e.g., peaks-and-valleys configuration).

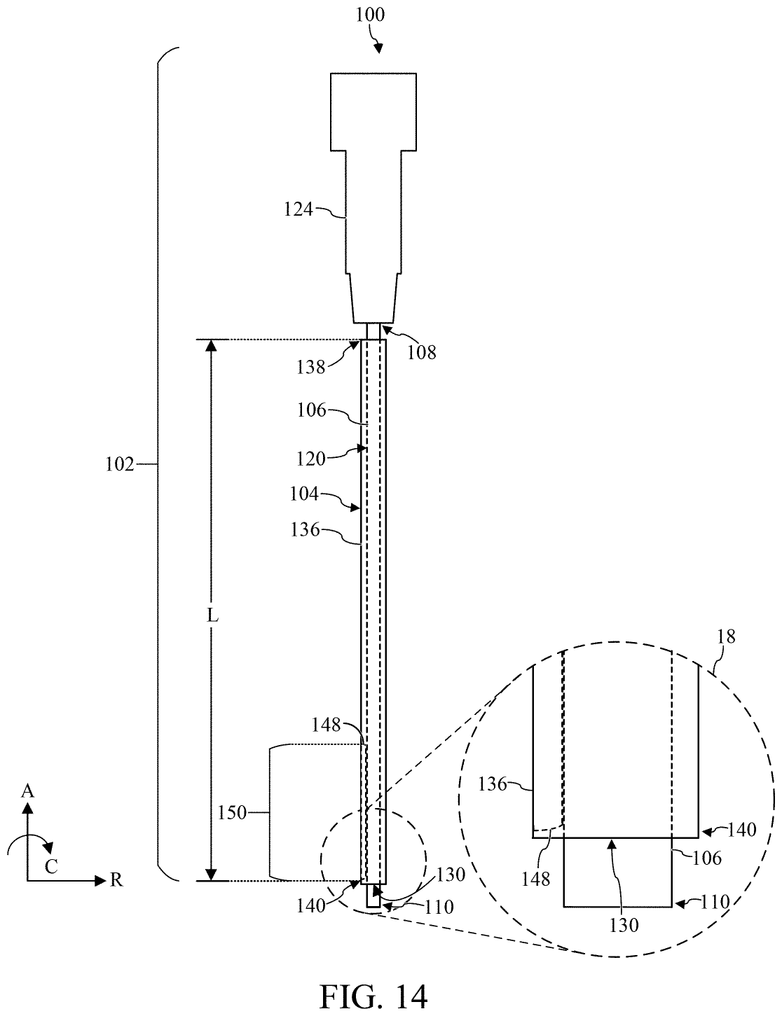

FIG. 14 shows another non-limiting example of endoscope system 100. In the example, access sheath 104 may include at least one projection 148 extending radially from inner surface 132 of conduit 136. As shown, conduit 136 may include a single projection 148 that extends over a portion 150 of conduit 136. That is, access sheath 104 may include a single projection 148 that extends radially from inner surface 132 of conduit 136 into opening 130, as well as extends axially over portion 150 of length (L) of conduit 136. In the non-limiting example, single projection 148 may be formed substantially adjacent second end 140 of conduit 136 and may extend axially toward and/or proximate first end 138. As similarly discussed herein, the size, length, and/or dimension of portion 150 in which projection 148 extends over conduit 136 may be dependent, at least in part, on the length (L) of conduit 130, the length (L) of shaft 106 of endoscope 102, the endoscopic procedure to be performed, and/or patient characteristics.

As shown in the non-limiting example, and similar to single protrusion 118 shown and discussed herein with respect to FIG. 13, single projection 148 may have a substantially uniform shape and/or configuration. As discussed herein, at least a portion of projection 148 may include a curved and/or non-linear surface to improve insertion and/or displacement of endoscope 102 into access sheath 104. Additionally, and as similarly discussed herein, single, uniform projection 148 may include and/or be formed with a predetermined (H) between approximately 0.1 millimeters (mm) and 1.5 mm. In other non-limiting examples (not shown), the height (H) of single projection 148 may vary over the axial length of projection 148. For example, projection 148 may include a central portion that includes a first height (H1), and two opposing end portions formed on opposing sides of central portion that include a second height (H2). The second height (H2) may be distinct (e.g., less than, greater than) from the first height (H1). The difference in high in each portion may be achieved via a stepped configuration in projection 148 or alternatively a gradual incline/decline between end portions and central portion. In another example (not shown), the height (H) of single projection 148 may vary multiple times over the axial length (e.g., peaks-and-valleys configuration).

FIG. 15 depicts example processes for performing an endoscopic procedure using an endoscope system including an endoscope and/or an access sheath. In some cases, the procedure may be performed using endoscope system 100, endoscope 102, and/or access sheath 104, as discussed above with respect to FIGS. 1-14. In embodiments, the method includes contacting a patient in need thereof with an endoscope system including endoscope and/or ureteral access sheath of the present disclosure, wherein the endoscope includes on or more protrusions as described herein to increase a flow of fluid out of the patient. In embodiments, the one or more protrusions are disposed in a predetermined position within the system to increase a flow of fluid out of the patient.

The process of performing the endoscopic procedure may include providing an endoscope system which may include an endoscope and/or a ureteral access sheath receiving the endoscope. The endoscope system may include at least one protrusion included within the shaft of the endoscope, and/or at least one feature included within the opening of the conduit of the access sheath. The feature(s) included within the opening of the conduit may to increase a flow of fluid at least one of through the working channel of the shaft, through the opening of the conduit and/or around the endoscope positioned within the opening of the conduit of the access sheath, and/or may reduce the intra-pelvic pressure experienced within the renal pelvis during the procedure.

The at least one protrusion included within the shaft of the endoscope may extend from an exterior surface of the shaft, and/or into the working channel. Additionally, the feature(s) included within the opening of the conduit of the access sheath includes at least one projection extending radially into the opening of the conduit, adjacent the shaft of the endoscope, and/or a non-circular or a non-concentric cross-sectional configuration for the opening of the conduit.

During the procedure the fluid is at least partially directed away from the surfaces not having the projection(s) included in the endoscope and/or the feature(s) included in the access sheath. Alternatively during the procedure, the fluid is at least partially directed towards the surfaces having the projection(s) included in the endoscope and/or the feature(s) included in the access sheath.

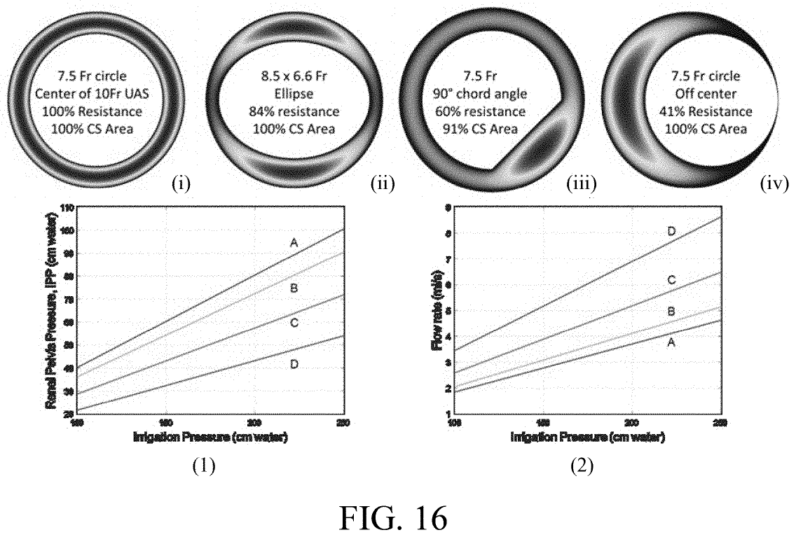

Referring to FIG. 16, an image of fluid resistance and cross-sectional area of for cross-sectional shapes of an opening in access sheath 104 for receiving endoscope 102 (i) a conventional opening 130 cross-sectional shape, (ii) an opening 130 with an elliptical cross-sectional shape, (iii) an opening 130 with a D-shaped cross-sectional area, (iv) the cross-sectional shape of an opening 130 being offset or non-concentric with outer surface 142 of access sheath 104.

Referring also to FIG. 16, two graphs showing (1) intra-pelvic pressure and (2) fluid flow rate (outflow) for various measurements of irrigation pressure for these four shapes is shown.

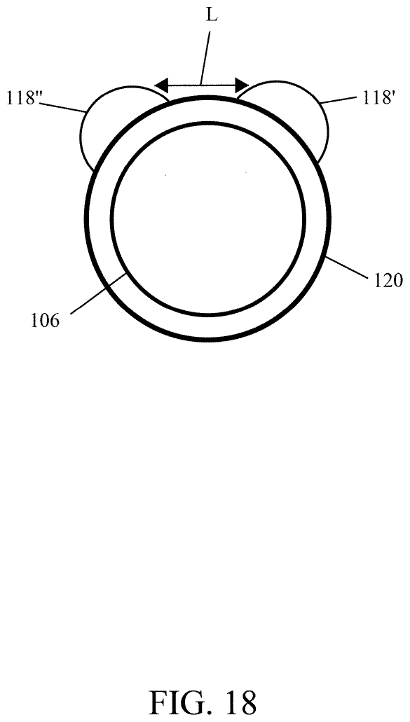

It is understood that the number of protrusions 118 and/or projections 148 shown herein on endoscope 102, shaft 106, and/or access sheath 104 is illustrative. As such and endoscope 102, shaft 106, and/or access sheath 104 of endoscope system 100 may include more or less protrusions 118/projections 148 than depicted in the non-limiting examples discussed herein. Furthermore, it is understood that the portion 122/150 including protrusions 118/projections 148 may span over or cover more or less of the length (L) of endoscope 102 and/or access sheath 104. For example, a plurality of protrusions 118 may span from first end 108 to second end 110 (e.g., entire length L) of endoscope 102. FIG. 18 shows two protrusions 118′ and 118″ in a cross-sectional view of a shaft 106.

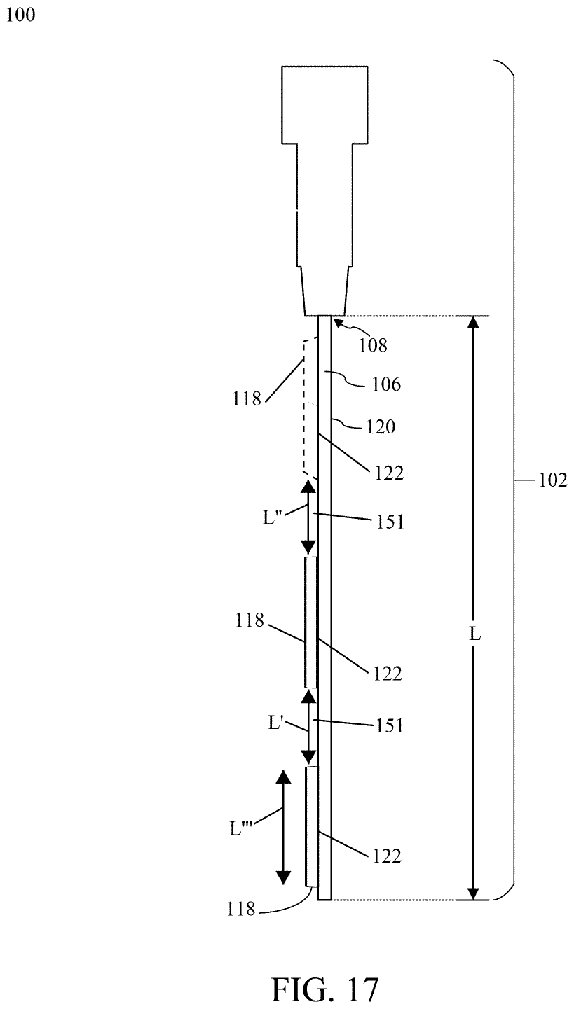

Referring to FIG. 17, another non-limiting example of endoscope system 100. More specifically, FIG. 17 shows another non-limiting example of endoscope 102 of endoscope system 100. As discussed in some examples herein, endoscope 102 may include at least one protrusion 118 (three shown) extending radially from exterior surface 120 of shaft 106. As shown, shaft 106 may include a plurality of protrusion 118 that extend over a portion 122 of shaft 106. That is, endoscope 102 may include a plurality or several protrusions 118 that extends radially from exterior surface 120 of shaft 106, as well as extends axially over portion 122 of length (L) of shaft 106. As similarly discussed herein, the size, length, and/or dimension of portion 122 in which protrusion 118 extends over shaft 106 may be dependent, at least in part, on the length (L) of shaft, the endoscopic procedure to be performed, and/or patient characteristics. In embodiments, the length of the protrusion, shown herein as L′″, may extend along a segment or portion 122 a top exterior surface 120. In embodiments, L′″ may be 0.1-20 cm, 0.1-10 cm, 2-20 mm, 2-10 mm, about 2 mm, about 3 mm, about 4 mm, 2 mm, 3 mm, or 4 mm. In embodiments, one or more gaps 151 are shown including a Length L′ and L″. In embodiments, L′ and L″ each be 0.1-20 cm, 0.1-10 cm, 2-20 mm, 2-10 mm, about 2 mm, about 3 mm about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, 2 mm, 3 mm 4 mm, 5 mm, 6 mm, 7 mm, 8 mm, 9 mm, 10 mm, 11 mm, 12 mm, 13 mm, 14 mm, 15 mm, 16 mm, 17 mm. In some embodiments, L′″ is 2 mm, and L′ and L″ are 2 mm. In embodiments, L′″ is 2 mm, and L′ and L″ are 4 mm. In embodiments, L′″ is 2 mm, and L′ and L″ are 8 mm. In embodiments, L′″ is 2 mm, and L′ and L″ are 10 mm. In embodiments, L′″ is 3 mm, and L′ and L″ are 3 mm. In embodiments, L′″ is 3 mm, and L′ and L″ are 6 mm. In embodiments, L′″ is 3 mm, and L′ and L″ are 9 mm. In embodiments, L′″ is 3 mm, and L′ and L″ are 12 mm. In embodiments, L′″ is 4 mm, and L′ and L″ are 4 mm. In embodiments, L′″ is 4 mm, and L′ and L″ are 8 mm. In embodiments, L′″ is 4 mm, and L′ and L″ are 12 mm. In embodiments, L′″ is 4 mm, and L′ and L″ are 16 mm.

FIG. 18 shows a cross-sectional view of a shaft 106 embodiment of the present disclosure including similar features as though shown and discussed herein with respect to FIGS. 1 and/or 2. Distinct from the embodiment shown in FIG. 1, protrusions 118′ and 118″ extend radially from exterior surface 120 of shaft 106 and are spaced apart by the predetermined distance (L). In embodiments, the space or distance between each of the plurality of protrusions 118 may vary. For example, a first protrusion 118′ and a second protrusion 118″ may be spaced a first predetermined distance (L). It is understood that the distance (L) separating each protrusion 118′ and 118″ of endoscope 102 may vary or be distinct in any suitable manner. In embodiments, protrusions 118′ and 118″ are disposed in a 10 o'clock and 2 o'clock position in accordance with the central axis of shaft 106. In embodiments, protrusions 118′ and 118″ are disposed in an 11 o'clock and 1 o'clock position in accordance with the central axis of shaft 106.

In embodiments, the present disclosure includes an endoscope shaft such as shaft 106 including: a first end, a second end positioned axial opposite of the first end, at least one working channel extending axially through the shaft, between the first end and the second end, and at least one protrusion extending at least one of: from an exterior surface of the shaft, or into the working channel. In embodiments, the endoscope shaft is fixedly attached to a handpiece releasably coupled to the shaft adjacent the first end. In embodiments, one or more protrusions extends along a length of the exterior surface 120 of the shaft 106 from the first end to the second end. In embodiments, one or more protrusions extend along one or more segments of the exterior surface of the shaft from the first end to the second end. In embodiments, the one or more segments extend along the same length of the exterior surface, such as in a line as shown in FIG. 17. Referring to FIG. 18, in one or more segments are shown extend along different lengths of the exterior surface such as over portion 122 and are separated by a variable or fixed or predetermined length (L). In embodiments, L may be a number between 0.1-30 mm, 0.1-20 mm, 0.1 to 10 mm, 0.1 to 5 mm, 1-5 mm, and the like.

In embodiments, the present disclosure includes an endoscope, for insertion into a ureter or an access sheath, the endoscope including: a shaft including: a first end, a second end positioned axial opposite of the first end, a working channel extending axially through the shaft, between the first end and the second end, and at least one protrusion extending at least one of: from an exterior surface of the shaft, or into the working channel; and a handpiece releasably coupled to the shaft adjacent the first end. In some embodiments, at least a portion of a surface of the at least one protrusion includes a curvature. In embodiments, at least one protrusion extends radially from the exterior surface of the shaft. In embodiments, the at least one protrusion includes a plurality of protrusions. In embodiments, each protrusion of the plurality of protrusions are axially aligned. In embodiments, each protrusion of the plurality of protrusions are axially spaced a predetermined distance from an adjacent protrusion. In embodiments, each protrusion of the plurality of protrusion is axially spaced between approximately one (1) centimeter (cm) and approximately 10 cm apart from the adjacent protrusion. In embodiments, the predetermined distance is based on a length of the shaft. In embodiments, a first protrusion of the plurality of protrusions is axially spaced a first predetermined distance from an adjacent, second protrusion, and wherein the second protrusion of the plurality of protrusions is axially spaced a second predetermined distance from an adjacent, third protrusion, the second predetermined distance distinct from the first predetermined distance. In embodiments, at least one protrusion is formed adjacent the second end of the shaft. In embodiments, the at least one protrusion includes a height between approximately 0.1 millimeters (mm) and approximately 1.5 mm. In embodiments, the at least one protrusion extends radially into the working channel of the shaft. In embodiments, the working channel of the shaft includes a non-circular configuration. In embodiments, the exterior surface of the shaft includes a non-circular cross-sectional configuration. In embodiments, the non-circular cross-sectional configuration of the exterior surface of the shaft includes one of: an elliptical cross-sectional configuration, or a segmented circular cross-sectional configuration including a chord.

In embodiments, the present disclosure includes an access sheath for receiving an endoscope, the access sheath including: a conduit including: a first end, a second end positioned opposite of the first end, an opening extended substantially between the first end and the second end, the opening receiving the endoscope, and at least one projection extending radially into the opening of the conduit to increase a flow of fluid at least one of through the opening of the conduit or around the endoscope positioned within the opening of the conduit. In embodiments, at least a portion of a surface of the at least one projection includes a curvature. In embodiments, the at least one projection extends radially from an inner surface of the opening of the conduit. In embodiments, the at least one projection includes a plurality of projections. In embodiments, each projection of the plurality of projections are axially aligned. In embodiments, each projection of the plurality of projections are axially spaced a predetermined distance from an adjacent projection. In embodiments, each projection of the plurality of projections is axially spaced between approximately one (1) centimeter (cm) and approximately 10 cm apart from the adjacent projection. In embodiments, the predetermined distance is based on a length of the conduit. In embodiments, a first projection of the plurality of projections is axially spaced a first predetermined distance from an adjacent, second projection, and wherein the second projection of the plurality of projection is axially spaced a second predetermined distance from an adjacent, third projection, the second predetermined distance distinct from the first predetermined distance. In embodiments, at least one projection is formed adjacent the second end of the conduit. In embodiments, the at least one projection includes a height between approximately 0.1 millimeters (mm) and approximately 1.5 mm.

In embodiments, the present disclosure includes an endoscope system including: an endoscope including: a shaft including: a first end, a second end positioned axial opposite of the first end, and a working channel extending axially through the shaft, between the first end and the second end; and a handpiece releasably coupled to the shaft of the endoscope adjacent the first end; an access sheath for receiving the endoscope, the access sheath including: a conduit including: a first end, a second end positioned opposite of the first end, and an opening extended substantially between the first end and the second end, the opening receiving the shaft of the endoscope; and at least one of: at least one protrusion included within the shaft of the endoscope, the at least one protrusion extending at least one of: from an exterior surface of the shaft, or into the working channel, or at least one projection extending radially into the opening of the conduit, wherein the at least one protrusion and the at least one projection increase a flow of fluid at least one of through the opening of the conduit or around the endoscope positioned within the opening of the conduit. In embodiments, the at least one protrusion of the shaft of the endoscope includes a single protrusion extending axially over a portion of the shaft. In embodiments, the at least one projection of the conduit of the access sheath includes a single projection extending axially over a portion of the conduit. In embodiments, the exterior surface of the shaft of the endoscope includes a non-circular cross-sectional configuration. In embodiments, the non-circular cross-sectional configuration of the exterior surface of the shaft of the endoscope includes one of: an elliptical cross-sectional configuration, or a segmented circular cross-sectional configuration including a chord.

In embodiments, the present disclosure includes a method of performing an endoscopic procedure, the method including: providing an endoscope system including at least one of an endoscope and a ureteral access sheath receiving the endoscope, wherein the endoscope system includes at least one of at least one protrusion included within a shaft of the endoscope, or at least one projection extending radially into an opening of a conduit of the ureteral access sheath, and wherein the at least one protrusion and the at least one projection increase a flow of fluid at least one of through the opening of the conduit or around the endoscope positioned within the opening of the conduit of the access sheath. In embodiments, the at least one protrusion included within the shaft of the endoscope extends at least one of: from an exterior surface of the shaft, or into a working channel extending through the shaft, and the at least one projection extending radially into the opening of the conduit, adjacent the shaft of the endoscope. In embodiments, the fluid is at least partially directed away from surfaces not having the at least one projection included in the endoscope or the at least one projection included in the access sheath. In embodiments, the fluid is at least partially directed towards surfaces having the at least one projection included in the endoscope or the at least one projection included in the access sheath.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. An endoscope, for insertion into a ureter or an access sheath, the endoscope comprising:
a shaft including:
a first end,
a second end positioned axial opposite of the first end,
a working channel extending axially through the shaft, between the first end and the second end,
at least one protrusion extending at least one of:
from an exterior surface of the shaft, or
into the working channel; and
a handpiece releasably coupled to the shaft adjacent the first end.

2. The endoscope of claim 1, wherein at least a portion of a surface of the at least one protrusion includes a curvature.

3. The endoscope of claim 1, wherein the at least one protrusion extends radially from the exterior surface of the shaft.

4. The endoscope of claim 1, wherein the at least one protrusion includes a plurality of protrusions.

5. The endoscope of claim 4, wherein each protrusion of the plurality of protrusions are axially aligned.

6. The endoscope of claim 4, wherein each protrusion of the plurality of protrusions are axially spaced a predetermined distance from an adjacent protrusion.

7. The endoscope of claim 6, wherein each protrusion of the plurality of protrusions is axially spaced between approximately one (1) centimeter (cm) and approximately 10 cm apart from the adjacent protrusion.

8. The endoscope of claim 6, wherein the predetermined distance is based on a length of the shaft.

9. The endoscope of claim 4, wherein a first protrusion of the plurality of protrusions is axially spaced a first predetermined distance from an adjacent, second protrusion, and wherein the second protrusion of the plurality of protrusions is axially spaced a second predetermined distance from an adjacent, third protrusion, the second predetermined distance distinct from the first predetermined distance.

10. The endoscope of claim 1, the at least one protrusion is formed adjacent the second end of the shaft.

11. The endoscope of claim 1, wherein the at least one protrusion includes a height between approximately 0.1 millimeters (mm) and approximately 1.5 mm.

12. The endoscope of claim 1, wherein the at least one protrusion extends radially into the working channel of the shaft.

13. The endoscope of claim 1, wherein the working channel of the shaft includes a non-circular configuration.

14. The endoscope of claim 1, wherein the exterior surface of the shaft includes a non-circular cross-sectional configuration.

15. The endoscope of claim 14, wherein the non-circular cross-sectional configuration of the exterior surface of the shaft includes one of:
an elliptical cross-sectional configuration, or
a segmented circular cross-sectional configuration including a chord.

16. An access sheath for receiving an endoscope, the access sheath comprising:
a conduit including:
a first end,
a second end positioned opposite of the first end,
an opening extended substantially between the first end and the second end, the opening receiving the endoscope, and
at least one projection extending radially into the opening of the conduit to increase a flow of fluid at least one of through the opening of the conduit or around the endoscope positioned within the opening of the conduit.

17. The access sheath of claim 16, wherein at least a portion of a surface of the at least one projection includes a curvature.

18. The access sheath of claim 16, wherein the at least one projection extends radially from an inner surface of the opening of the conduit.

19. An endoscope system comprising:
an endoscope including:
a shaft including:
a first end,
a second end positioned axial opposite of the first end, and
a working channel extending axially through the shaft, between the first end and the second end; and
a handpiece releasably coupled to the shaft of the endoscope adjacent the first end;
an access sheath for receiving the endoscope, the access sheath including:
a conduit including:
a first end,
a second end positioned opposite of the first end, and
an opening extended substantially between the first end and the second end, the opening receiving the shaft of the endoscope; and
at least one of:
at least one protrusion included within the shaft of the endoscope, the at least one protrusion extending at least one of:
from an exterior surface of the shaft, or
into the working channel, or
at least one projection extending radially into the opening of the conduit,
wherein the at least one protrusion and the at least one projection increase a flow of fluid at least one of through the opening of the conduit or around the endoscope positioned within the opening of the conduit.

20. The endoscope system of claim 19, wherein the at least one protrusion of the shaft of the endoscope includes a single protrusion extending axially over a portion of the shaft.
